(19) United States
(12) Patent Application Publication
(10) Pub. No.: US 2026/0354906 A1
(43) Pub. Date: Nov. 12, 2026

(54) System and method for implanting a prosthesis, in particular in the glenoid cavity of a scapula

(71) Applicant: FX SHOULDER SOLUTIONS, Viriat (FR)

(72) Inventor: Baptiste Martin, Viriat (FR)

(73) Assignee: FX SHOULDER SOLUTIONS, Variat (FR)

(21) Appl. No.: 19/667,743

(22) Filed: May 5, 2026

(30) Foreign Application Priority Data
May 5, 2025 (FR) FR2504725
Oct. 14, 2025 (FR) FR2511881

(51) Int. Cl. A61B 34/20; A61B 17/16; A61B 34/10

(52) U.S. Cl. CPC A61B 34/20; A61B 17/1659; A61B 34/10; A61B 17/1684; A61B 2034/102; A61B 2034/107; A61B 2034/2065

(57) ABSTRACT

A system for installing a part of a prosthesis on a bone including a reamer provided to implant an element of the prosthesis; an augmented reality mask for capturing in real time a spatial position of the bone and a spatial position of the implantation device; a computer system for storing a digital model of the prosthesis on the bone as well as for creating in real time a combined image of the model and of the relative positions of the bone and of the implantation device; the mask also allowing an operator of the implantation to view in real time the combined image.

### TECHNICAL FIELD

The present invention relates to the field of joint prostheses; it relates in particular to a system and a method for implanting a glenohumeral joint prosthesis of a shoulder, between the scapula and the humerus. More particularly, the invention relates to a system and a method for installing a joint implant replacing the glenoid cavity.

It is particularly difficult to install an implant replacing the glenoid cavity. Most of the scapula is in fact thin; it is not easy to install an implant in this reduced thickness. It is therefore important to preserve as much as possible the bone mass surrounding the glenoid cavity to install this implant efficiently.

### BACKGROUND ART

Before installing the implant, it is current practice to remove the labrum and ream the glenoid cavity to create a support for the implant. This reaming is carried out to remove the cartilage and sufficient bone. The bone is ground down until it bleeds, to freshen it, so that the cells colonize the implant and guarantee its sealing. According to a process of the prior art, reaming is carried out by an operator, who is typically a surgeon, by iteration, until they obtain the required profile. However, this process relies mainly on the experience of the operator and their perception, more or less complete and precise, of the characteristics of the glenoid cavity that they are reaming. The iterative process is intrinsically long. In addition, this process, which is less precise, could remove more bone than necessary, whereas as much bone as possible should be preserved in a scapula which naturally has low bone stock.

### SUMMARY OF THE INVENTION

One objective of the invention is to propose means to perform a faster and more precise implantation and, in particular to provide a solution that can reproduce on the patient an intervention that has been planned virtually.

According to the invention, a system for installing a part of a prosthesis on a bone comprises:
means for storing a digital model of said bone and of said prosthesis on said bone;
means provided to implant an element of said prosthesis;
means for capturing in real time a spatial position of the bone;
means for capturing in real time a spatial position of said implantation means;
means for creating in real time a combined image of the model and of the relative positions of the bone and of the implantation means; and,
means (V) for allowing an operator (P) of the implantation to view in real time said combined image.

The implantation means can comprise a reamer.

Augmented reality means, preferably augmented reality headset, mask (V) or glasses advantageously comprise:
the means for capturing in real time a spatial position of the bone;
the means for capturing in real time a spatial position of said implantation means; and,
the means allowing an operator of the implantation to view in real time said combined image.

The implantation means advantageously comprise a reference preferably comprising at least one graphic code, preferably of the QR-code type, said reference being adapted in order to determine the spatial position of the implantation means.

### BRIEF DESCRIPTION OF THE DRAWINGS

Embodiments and variants will be described below, given as non-limiting examples, and referring to the attached drawings in which:

FIG. 1A shows a first view along a horizontal direction of a right scapula of a human being;

FIG. 1B shows a second view along a horizontal direction of a right scapula of a human being;

FIG. 2A shows a first view along a direction of the glenoid cavity of the scapula of FIG. 1;

FIG. 2B shows a second view along a direction of the glenoid cavity of the scapula of FIG. 1

FIG. 3 shows a cross-sectional view of a reverse prosthesis for a shoulder joint for the scapula of FIG. 1;

FIG. 4 shows an installation of a pin using an electric screwdriver equipped with a lateral spatial reference; and,

FIG. 5 shows a reamer equipped with a posterior spatial reference.

### INVENTION

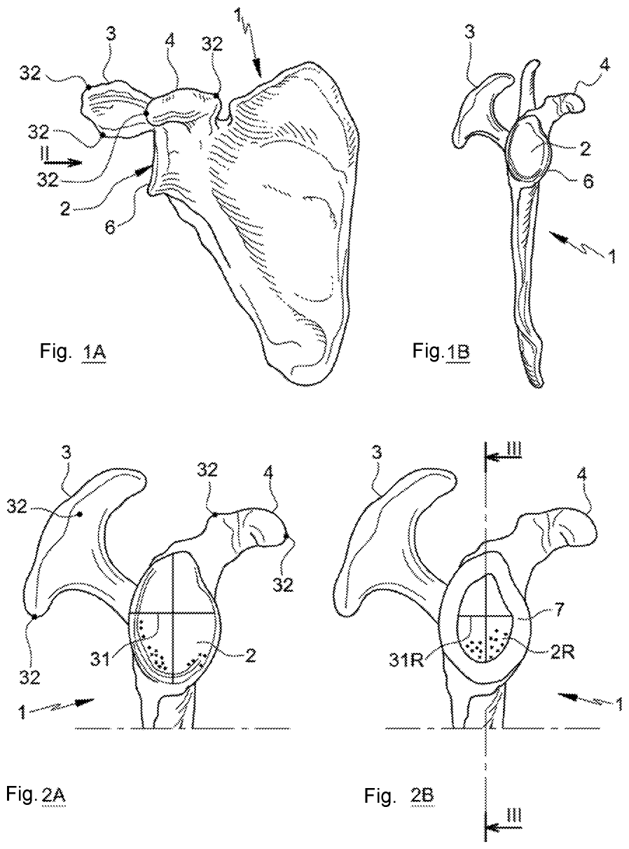

FIGS. 1 to 3 are all elevation views, in other words the top of the figure corresponds to the top of the body of the human being, potentially the patient, whose scapula is shown, when this human being is standing. The expression “right scapula” refers to the actual right side of this human being.

FIG. 1 shows the scapula 1 of a right shoulder. The figure includes two views 1A, 1B of the scapula. View 1A, on the left of FIG. 1, shows the scapula 1 from the front; view 1B, on the right of FIG. 1, shows the scapula 1 from the side, with the glenoid cavity 2 facing the observer. The acromion 3 and the coracoid 4 can also be seen on the views of FIG. 1.

FIG. 2 shows more particularly the glenoid cavity 2. This figure includes two views 2A, 2B, taken along the direction II of FIG. 1A. On the right of FIG. 2, view 2A shows the glenoid cavity 2 in its initial configuration, before reaming; on the left of FIG. 2, view 2B shows the glenoid cavity 2 after reaming.

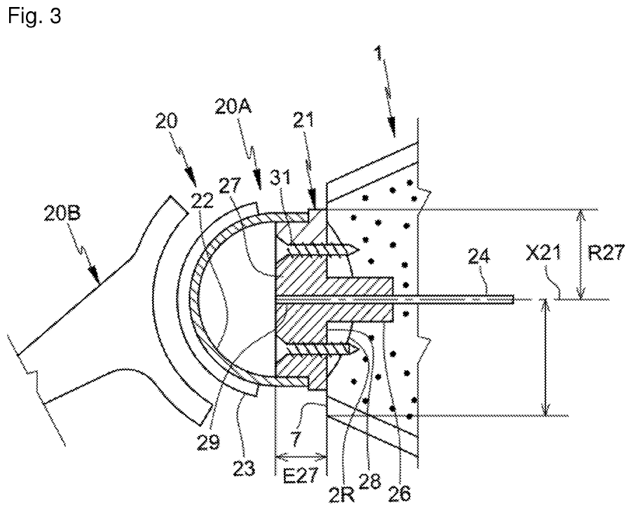

The glenoid cavity 2 has the shape of a cup, concave, surrounded by an annular peripheral labrum 6. The method according to the invention comprises a phase of reaming the crest 6 to create a support 7 in order to place therein an insert 21 of a glenoid cavity implant of a joint prosthesis 20, as shown on FIG. 3. The support 7 has the shape of an annular plate around the residual part 2R of the glenoid cavity 2. The prosthesis is not shown on FIG. 2.

FIG. 3 shows a cross-section of the prosthesis 20, along the cross-sectional plane III-III of view 2B. The prosthesis 20 comprises a scapular part 20A and a humeral part 20B. The scapular part 20A is attached to the scapula. The humeral part 20B is attached to the humerus. The two parts 20A and 20B form together a ball-and-socket type joint which recreates the joint function of the shoulder, between the scapula and the humerus. The prosthesis 20 is a reverse prosthesis, in other words the scapular part 20A has a convex shape, while the humeral part has a concave shape, the shapes cooperating to create a ball-and-socket joint.

The scapular part 20A comprises the insert 21, a hemispherical bulb 22 (or glenosphere), a cup 23 and a pin 24. FIG. 3 is a diagrammatic, cross-sectional view showing the arrangement of these elements 21-24, when the prosthesis 20 is in the functional position.

The insert has the shape of a mushroom of revolution about an insert axis X21; it comprises a rod 26 and a head 27. The head has the shape of a disc E27 that is thin compared with its radius R27. The rod 26 extends axially from an internal side 28 of the head. An axial passage 29 is drilled through the insert 21 to receive the pin 24 and attach this pin to the insert 21. Screws 31 are provided to attach the head 27 so that the internal side 28 is pressed against the support 7. In the example shown, four screws are used, two of which are shown on FIG. 3.

The bulb 22 is attached to the head of the insert, which acts as a bracket. The cup 23 covers the bulb and reduces the friction between the scapular part 20A and the humeral part 20B of the joint 20. Typically, the bulb 22 has a diameter of about twenty-four millimeters.

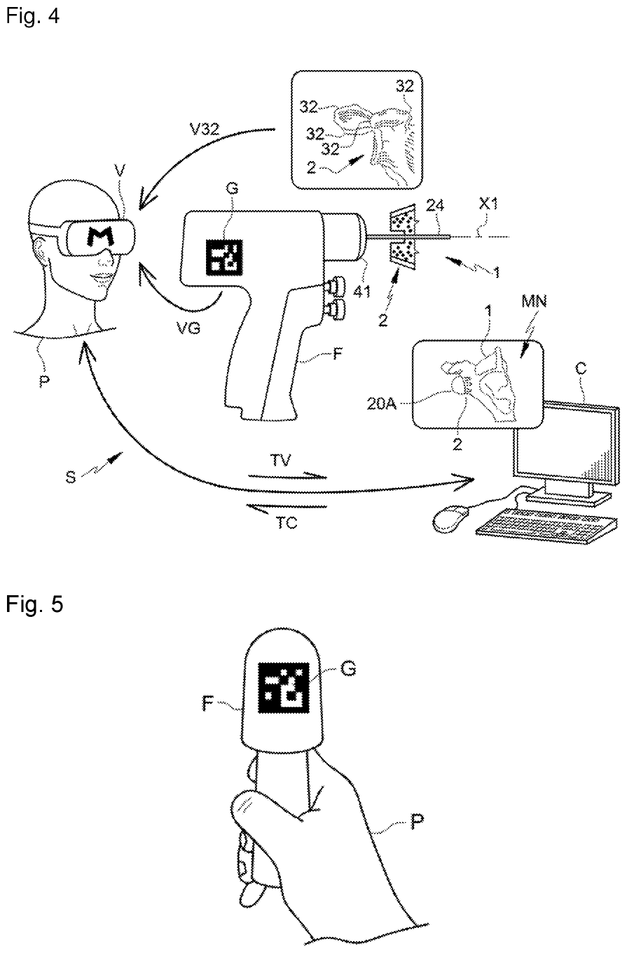

In a method according to the invention for preparing the phase of reaming the support 7, a three-dimensional (3D) digital model MN of the patient's shoulder is first produced. This model is produced using medical imaging means. These imaging means can be of the scanner and/or MRI type. These means provide points distributed in several two-dimensional (2D) images; the 2D images are compiled to form a 3D image consisting of a point cloud, this point cloud being an accumulation of points of the compiled 2D images. The model of the patient's shoulder is obtained from the 3D image thus formed. The model identifies the various constituents present on the image, in particular the cartilages and the bone of the scapula, in particular at the glenoid cavity. This step also applies for the installation of some elements of the prosthesis. This is the case in particular for the installation of the pin 24 in the glenoid cavity, as will be described referring to FIG. 4 et 5, prior to the reaming phase, the reaming step using the pin to guide it along the pin, in the direction of the depth, only the reaming depth still having to be adjusted.

In this method for preparing the reaming phase, computer processing means C are used to automatically define the optimum reaming to be produced. This reaming must create a support 7 with no cartilage and the bone must be sufficiently freshened to guarantee the sealing of the insert 21, without removing too much bone. These means for automatic definition of optimum reaming are advantageously trained automatically, using data from previous reaming operations, in particular carried out iteratively as explained above. It can also be corrected by the operator, before or during the reaming operation and can learn from these corrections.

A reamer is used for the reaming operations. This reamer can be held and used manually by the operator. The reaming can be automated and the reamer can be handled by a robot which uses instructions generated by the computer means based on the optimum reaming defined previously. The reamer is not shown on the figures.

A first reference 31, 32 (shown on FIG. 2) is also defined on the surface of the glenoid cavity and viewing means V are used to check the progress of the reaming operation, compared with the predefined optimum reaming.

In particular when the operator uses a manual reamer, the reamer has its own second reference, such that the viewing means can determine a relative position of the first reference 31, 32 and of the second reference. The viewing means can advantageously comprise augmented reality means, for example augmented reality headset or glasses V (see FIG. 4), so that the operator can view in real time the progress of the reaming. The second reference can be one or more graphic codes G applied on the reamer F. This graphic code can be of the QR-Code G type or similar.

We will now describe a first type of first reference, referring to FIG. 2.

As shown on view 2A of FIG. 2, the first type can be a first reference 31 that can be marked by the operator across the glenoid cavity 2. In the example shown, this first reference has the shape of a cross, when the glenoid cavity is seen from the front. During the reaming operation, the material is removed with the portions of the first reference 31 which were marked. After the reaming, as shown on view 2A, only a residual part 31A of the first reference 31 is still visible on the residual part 2R of the glenoid cavity. The progressive disappearance of the first reference 31 indicates the progress of the reaming. The reaming can be carried out under the center of the cross, preferably at a distance substantially equal to twelve millimeters from the center of the cross.

We will now describe a second type of first reference, referring to FIGS. 1 and 2.

As shown on views 1A and 2A, the second type of first reference consists of several characteristic points 32, distributed on the patient's scapula. In the example shown, there are two points 32 on the acromion 3 and two other points 32 on the coracoid 4. A sufficient number of these points is chosen to form a reference that can be seen by the viewing means throughout the reaming phase. The reference points 32 can comprise points chosen automatically by the computer means in the point cloud of the model; they can also comprise points marked physically by the operator on the scapula 1.

Obviously, the two types of first reference can be used simultaneously.

Referring to FIG. 4, we will now describe a method for installing a prosthesis 20 according to the invention in that it can be used to position a pin 24 in order to install a prosthesis 20A on the glenoid cavity 2 of a scapula 1, as well as a system S to allow the implementation of such a method by an operator P. Typically, the operator P is a surgeon.

The system S comprises:
a reamer F, having substantially the shape of a gun;
a computer system C;
viewing means V, in this case an augmented reality mask V.

A digital model MN of the scapula 1 and of the prosthesis 20A considered has been produced and saved in the computer system C.

In the configuration of FIG. 4, the reamer F is in the drilling position, the pin 24 being placed in the chuck 41 of the reamer. The operator P uses the reamer F to position the pin 24 in the scapula 1 through the glenoid cavity 2, along the predefined axis X21 of the prosthesis 20A.

The reamer F is marked with a graphic code G. In the example of FIG. 4, the graphic code is a QR code; it is placed on the right side of the reamer F. In the example of FIG. 5, the code G is placed on the back of the reamer F. Several codes can be placed at different positions of the reamer so that at least one of them can be seen by the operator P.

As shown on FIG. 4, the operator P wears the mask V. This mask V is used to see and digitize V32 the references, in this case the references 32 previously placed on the scapula. The mask V can also be used to see and digitize VG the reference G of the reamer F. The information thus collected is transmitted TV by the mask V to the computer system C. The computer system combines the information received with the model MN in order to create an augmented image M showing the relative positions of the scapula 1, of the pin 24 being positioned and the theoretical position of the pin obtained from the model MN. The image M thus created is projected in real time in the mask V, so that the operator P can ensure at all times that the position of the pin 24 corresponds substantially to the ideal position determined.

Although this method has been illustrated referring to the installation of the pin 24, it could also be used to ream the glenoid cavity, in other words to create the support 7. It can also be used to install the screws 31.

Obviously, the invention is not limited to the examples which have just been described. On the contrary, the invention is defined by the following claims.

It will appear to those skilled in the art that various modifications can be made to the embodiments described above, in the light of the information that has just been disclosed.

In fact, in an installation method according to the invention, it is possible not to use artificial references placed on the scapula to determine a position of the scapular in space. To do so, the shape of the scapula itself can be used.

This invention is particularly advantageous. During an operation, in fact, the glenoid cavity is difficult to access, in particular to be seen by the operator, at the bottom of a hole where the soft parts obstruct the view.

1. A system for installing a part of a prosthesis on a bone, comprising:
a storing device for storing a digital model of said bone and of said prosthesis on said bone;
an implant device to implant an element of said prosthesis;
a capturing device for capturing in real time a spatial position of the bone;
a real time capturing device for capturing in real time a spatial position of said implantation device;
a combination device for creating in real time a combined image of the model and of the relative positions of the bone and of the implantation device; and,
an implantation device to allow an operator of the implantation to view in real time said combined image.

2. The system according to claim 1, wherein the implantation device comprise a reamer.

3. The system according to claim 1, wherein an augmented reality device such as an augmented reality headset, a mask, or glasses comprises: the device for capturing in real time the spatial position of the bone; the device for capturing in real time the spatial position of said implantation device; and the device for allowing the operator of the implantation to view in real time said combined image.

4. The system according to claim 1, wherein the implantation device comprises a marker comprising at least one graphic code, such as a QR-code type, said marker being adapted in order to determine the spatial position of said implantation device.
